(19) United States
(12) Patent Application Publication
(10) Pub. No.: US 2010/0082054 A1
(43) Pub. Date: Apr. 1, 2010

(54) FLUID EJECTION DEVICE AND FLUID EJECTION METHOD

(75) Inventors: Takeshi SETO, Chobu-shi (JP); Kazuo KAWASUMI, Chino-shi (JP); Yasuyoshi HAMA, Shimosuwa-machi (JP); Hideki KOJIMA, Matsumoto-shi (JP); Yasuhiro ONO, Matsumoto-shi (JP)

Correspondence Address: OLIFF & BERRIDGE, PLC, P.O. BOX 320850, ALEXANDRIA, VA, 22320-4850, US

(73) Assignee: SEIKO EPSON CORPORATION, Tokyo (JP)

(21) Appl. No.: 12/568,161

(22) Filed: Sep. 28, 2009

(30) Foreign Application Priority Data
Oct. 1, 2008 (JP) 2008-256093

(51) Int. Cl. A61B 17/32

(52) U.S. Cl. 606/167

(57) ABSTRACT

A fluid ejection device includes: a fluid chamber in which fluid flows; a volume varying unit having a pressure generating element that varies the volume of the fluid chamber on the basis of a driving signal; an inlet fluid channel and an outlet fluid channel intercommunicating with the fluid chamber; a fluid channel pipe having a connection fluid channel that intercommunicates with the outlet fluid channel at a first end thereof and intercommunicates with a fluid ejection port at a second end thereof; a fluid supply unit that supplies fluid to the inlet fluid channel; and a controller that applies a driving signal to the pressure generating element and controls variation of the volume of the fluid chamber by the volume varying unit, wherein the controller applies a driving signal to the pressure generating element so that a maximum ejection velocity of fluid in liquid is larger than a maximum ejection velocity of fluid in the air.

Japanese Patent Application No. 2008-256093 filed on Oct. 1, 2008, is hereby incorporated by reference in its entirety.

### BACKGROUND

1. Technical Field

The present invention relates to a fluid ejection device, a fluid ejection method and a surgical instrument that can incise or cut out a target site by ejecting fluid to the target site.

2. Related Art

A fluid technique in which a target site is incised, cut out or the like by ejecting fluid to the target site is hitherto known. For example, such a fluid ejection device as disclosed in JP-A-2008-82202 is known as a fluid ejection device for incising or cutting out a biomedical tissue in a medical field. This fluid ejection device includes a micro pump for varying the volume of a pump chamber by a piezoelectric element to execute a fluid discharging operation, a connection fluid channel which is connected to an outlet fluid channel of the micro pump at one end portion thereof and also provided with an opening portion (nozzle) smaller than the diameter of the outlet fluid channel at the other end portion thereof, and a connection pipe having rigidity in which the connection fluid channel is formed and pulsation of fluid flowing from the micro pump is transmissible to the opening portion. In this fluid ejection device, the fluid is made to flow while a pulsating wave group and a recess portion are repeated, and ejected from the opening portion at high velocity.

According to the technique disclosed in JP-A-2008-82202, pulsating fluid can be ejected at high velocity, and also it can be easily controlled. Furthermore, ejection of pulsating fluid has high incision performance in a surgical operation or the like, and also the flow amount of fluid may be small, so that fluid is little trapped in an operative field. Accordingly, this technique has an effect of enhancing visibility and preventing spattering of tissues.

With the inclusion of the technique disclosed in JP-A-2008-82202, it is assumed in a fluid ejection device for incising or cutting out a target site by ejecting fluid that a unit (handpiece) for ejecting fluid is used in close contact with the target site in order to perform a minute work.

However, when the fluid ejection device as described above is used for a surgical operation, it is substantially extremely difficult for an operator to continue the surgical operation with keeping the contact between the nozzle of the handpiece and the target site so that a surgery site is prevented from being damaged during the surgical operation. Accordingly, in the fluid ejection device, the fluid ejection may be executed under a state that the nozzle is not in close contact with the target site.

In such a case, the ejection is carried out at the same level in both a close-contact state and a disjunction (non-contact) state in the fluid ejection device. Therefore, there may occur such a situation that fluid spatters to a surrounding area or fluid is ejected to an unexpected site, so that the site concerned is erroneously cut out. That is, with respect to the fluid ejection device in which a target site is incised or cut out by ejecting fluid to the target site, it has been desired to control discharge of fluid more properly.

### SUMMARY

An advantage of some aspects of the invention is to more properly perform discharge control of fluid in a fluid ejection device which incises or cuts out a target site by ejecting fluid to the target site.

A fluid ejection device according to a first aspect of the invention includes: a fluid chamber (for example, fluid chamber 501 of FIG. 2A) in which fluid flows; a volume varying unit (for example, piezoelectric element 401 and diaphragm 400 of FIGS. 2A and 2B) having a pressure generating element that varies the volume of the fluid chamber on the basis of a driving signal; an inlet fluid channel and an outlet fluid channel intercommunicating with the fluid chamber; a fluid channel pipe (for example, connection fluid channel pipe 200 of FIGS. 2A and 2B) having a connection fluid channel that intercommunicates with the outlet fluid channel at a first end thereof and intercommunicates with a fluid ejection port at a second end thereof; a fluid supply unit (for example, fluid container 10 and pump 20 of FIG. 1) that supplies fluid to the inlet fluid channel; and a controller (for example, controller 30 of FIG. 4) that applies a driving signal to the pressure generating element and controls variation of the volume of the fluid chamber by the volume varying unit, wherein the controller applies a driving signal to the pressure generating element so that a maximum ejection velocity of fluid in liquid is larger than a maximum ejection velocity of fluid in the air.

According to the above construction, when the tip of the fluid channel pipe is in the liquid, the fluid is ejected at a higher ejection velocity, and when the tip of the fluid channel pipe is in the air, the fluid is ejected at a lower ejection velocity. Accordingly, even when fluid is ejected under a state that a nozzle is not in close contact with an incision or cut-out target site, the fluid is ejected at a lower ejection velocity, so that an effect when the fluid spatters to the surrounding area or the fluid is ejected to an unexpected site can be suppressed. That is, when fluid is ejected to a target site to incise or cut out the target site, the discharge control of the fluid can be more properly performed.

It is preferable that the controller applies as the driving signal voltage pulse waveforms whose period is set so that the maximum ejection velocity of fluid in liquid is larger than the maximum ejection velocity of fluid in the air. With the construction as described above, by setting the period of the driving signal in conformity with a condition, the ejection velocities in the air and the liquid can be properly set.

Furthermore, it is preferable that the controller applies to the pressure generating element the driving signal with which the maximum ejection velocity of the fluid in the air is less than 20 meters per second. With the construction as described above, even when the fluid is ejected in the air, the fluid is ejected to the extent that the ejected fluid does not incise or cut out a biomedical tissue. Therefore, when a target site is incised or cut out by ejecting fluid, the discharge control of the fluid can be more properly performed.

A fluid ejection device according to a second aspect of the invention includes: a fluid chamber (for example, fluid chamber 501 of FIG. 2A) in which fluid flows; a volume varying unit (for example, piezoelectric element 401 and diaphragm 400 of FIGS. 2A and 2B) having a pressure generating element that varies the volume of the fluid chamber on the basis of a driving signal; an inlet fluid channel and an outlet fluid channel intercommunicating with the fluid chamber; a fluid channel pipe (for example, connection fluid channel pipe 200 of FIGS. 2A and 2B) having a connection fluid channel that intercommunicates with the outlet fluid channel at a first end thereof and intercommunicates with a fluid ejection port at a second end thereof; a fluid supply unit (for example, fluid container 10 and pump 20 of FIG. 1) that supplies fluid to the inlet fluid channel; and a controller (for example, controller 30 of FIG. 4) that applies a driving signal to the pressure generating element and controls variation of the volume of the fluid chamber by the volume varying unit, wherein the controller applies a driving signal to the pulsation generator so that fluid and gas are mixed with each other to form a gas-liquid mixture portion in the connection fluid channel pipe due to a fluid discharge executed immediately before the fluid is discharged in the air, and the fluid discharge is subsequently executed under a state that the gas-liquid mixture portion remains (for example, the state of a time t=t4 in FIG. 7B).

With the construction described above, when fluid is discharged in the air, a subsequent pulsation wave reaches the tip portion of the fluid channel pipe under the state that the gas-liquid mixture portion exists in the fluid channel pipe. Accordingly, as compared with a case where no gas-liquid mixture portion exists, the fluid is ejected at a lower ejection velocity. Accordingly, even when fluid is discharged under the state that the nozzle is not in close contact with the incision or cut-out target site (that is, under the non-liquid state), the fluid is merely ejected at a lower ejection velocity. Therefore, the effect when fluid spatters to a surrounding area or fluid is ejected to an unexpected site can be suppressed. That is, when the target site is incised or cut out by ejecting the fluid to the target site, the fluid discharge control can be more properly performed.

Furthermore, it is also preferable that the controller applies the driving signal so that the gas-liquid mixture portion is formed at the back side of the fluid ejection port of the connection fluid channel pipe when the fluid is discharged in the air. With the construction as described above, when pulsed fluid is discharged from the fluid channel pipe, a gas-liquid mixture portion is formed in a space in the fluid-discharged fluid channel pipe with following the pulsed fluid. Therefore, the fluid discharge control can be more properly performed by simple control.

A fluid ejection method according to a third aspect of the invention includes: ejecting fluid to which pressure is applied by a pressure generating element so that a maximum ejection velocity of the fluid in liquid is larger than a maximum ejection velocity of the fluid in the air when the fluid is ejected through a fluid channel from a fluid ejection port. According to this method, even when the fluid is discharged under a state that a nozzle is not in close contact with an incision or cut-out target site, the fluid is merely ejected at a lower ejection velocity. Therefore, the effect when the fluid spatters to a surrounding area or the fluid is ejected to an unexpected site can be suppressed. That is, when the target site is incised or cut out by ejecting the fluid, the fluid discharge control can be more properly performed.

As described above, according to the aspects of the invention, the fluid discharge control can be more properly performed in the fluid ejection device that can incise or cut out the target site by ejecting the fluid.

### BRIEF DESCRIPTION OF THE DRAWINGS

The invention will be described with reference to the accompanying drawings, wherein like numbers reference like elements.

FIG. 1 is a diagram showing the construction of a fluid ejection device according to a first embodiment of the invention.

FIGS. 2A and 2B are diagrams showing the structure of a pulsation generator according to the first embodiment.

FIG. 3 is a plan view showing an example of an inlet fluid channel.

FIG. 4 is a functional block diagram showing a control system of the fluid ejection device.

FIG. 5 is a schematic diagram showing a voltage pulse waveform stored in a waveform storing unit.

FIG. 6 is a flowchart showing discharge control processing executed by a signal controller.

FIGS. 7A to 7C are diagrams showing the state of a connection fluid channel pipe when fluid is ejected.

### DESCRIPTION OF EXEMPLARY EMBODIMENTS

Embodiments of the invention will be described hereunder with reference to the accompanying drawings.

The embodiments described below are preferable examples of the invention, and thus various limitations which are technically preferable are added to these embodiments.

However, the scope of the invention is not limited to these embodiments unless there is any description particularly limiting this invention in the following description.

Furthermore, the figures to be referred to in the following description are schematic diagrams showing members or parts at different scale sizes from those of the actual members or parts for convenience's sake of illustrations.

A fluid ejection device according to the invention is applicable to various matters such as drawing using ink or the like, cleaning of minute objects and structures, cutting, cut-out, incision, etc. of objects, a water pulse surgical knife for surgical operation. However, in the following embodiments, the fluid ejection device is typically applied as a surgical instrument which is suitably used to incise or cut out a biomedical tissue. Accordingly, fluid used in the following embodiments is water or normal saline solution.

### First Embodiment

On the basis of the viewpoint that the ejection velocity of fluid in a fluid ejecting operation is different between ejection of fluid in the air and ejection of fluid in liquid in accordance with the driving style of a piezoelectric element in some cases, a fluid ejection device according to a first embodiment is designed to drive a piezoelectric element so that the fluid ejection velocity is set to a low value in the air and a high value in liquid. By driving the piezoelectric element as described above, fluid discharge (ejection) control can be more properly performed in a fluid ejection device that can incise or cut out a target site (surgery target site) by ejecting the fluid to the target site.

### Construction

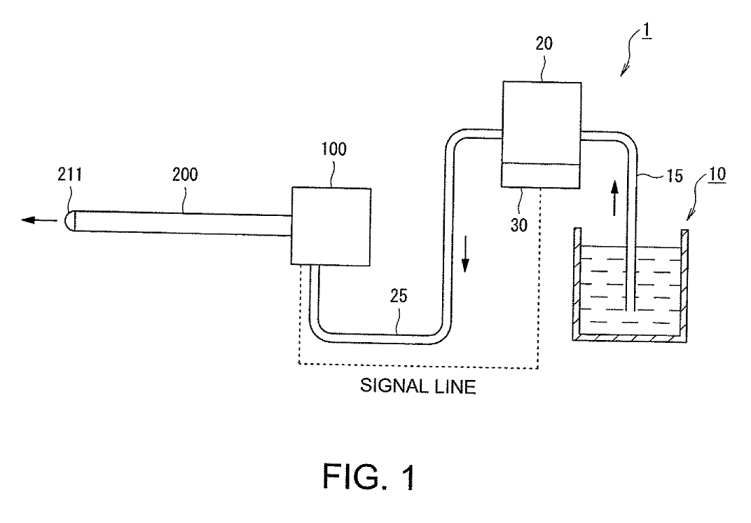

FIG. 1 is a diagram showing the construction of the fluid ejection device 1 according to the first embodiment.

In FIG. 1, the fluid ejection device 1 has, as basic constituent elements, a fluid container 10 in which fluid is stocked, a pump 20 for generating fixed pressure and supplying fluid to a pulsation generator 100, a controller 30 for controlling ejection of fluid, and the pulsation generator 100 for pulsating the fluid supplied from the pump 20 and making the pulsated fluid flow. In this embodiment, the controller 30 and the pump 20 are constructed as an integral unit, and the controller 30 and the pulsation generator 100 are connected to each other through a signal line for inputting/outputting various kinds of signals.

### Construction of Fluid Channel

First, the construction of the fluid channel of the fluid ejection device 1 will be described.

In this embodiment, the pulsation generator 100 constitutes a fluid ejection unit for ejecting fluid with pressure generated by a piezoelectric element. The fluid ejection unit corresponds to a handpiece which is gripped and used by an operator during a surgical operation or the like when the fluid ejection device 1 is constructed as a water pulse surgical knife. A narrow pipe-shaped connection fluid channel pipe 200 is connected to the pulsation generator 100, and a nozzle 211 having a size-reduced fluid channel therein is inserted in the tip portion of the connection fluid channel pipe 200.

The flow of fluid in the fluid ejection device 1 will be briefly described.

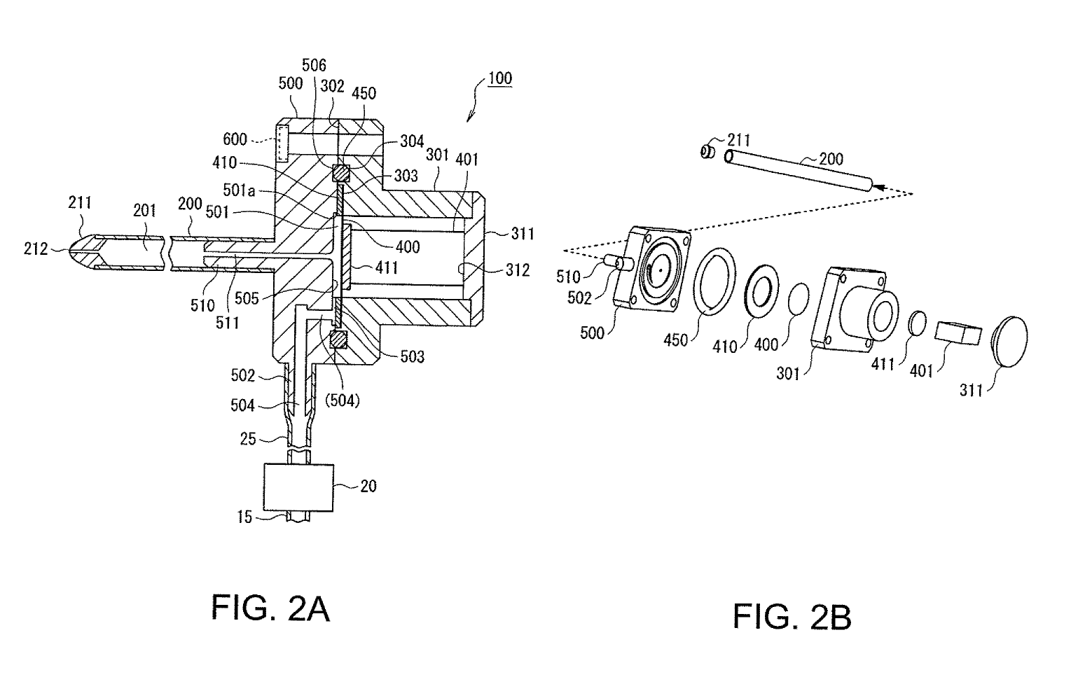

Fluid stocked in the fluid container 10 is sucked through a connection tube 15 by the pump 20, and supplied to the pulsation generator 100 through a connection tube 25 under fixed pressure. The pulsation generator 100 has a fluid chamber 501 (see FIG. 2A) and a volume varying unit for the fluid chamber 501, and it drives the volume varying unit to generate pulsation and eject fluid through the connection fluid channel pipe 200 and the nozzle 211 at high velocity. The details of the pulsation generator 100 will be described later with reference to FIGS. 2A, 23 and 3.

A unit for implementing the function of generating pressure to supply fluid from the fluid container 10 is not limited to the pump 20, and various styles may be used. For example, an infusion solution bag may be held at a higher position than the pulsation generator 100 by a stand or the like to generate supply pressure. Or, fluid may be sucked from the fluid container 10 to the fluid chamber 501 by using the pulsating action of discharging fluid through the volume varying unit of the pulsation generator 100. In these cases, there is an advantage that the pump 20 is not required and thus the construction can be simplified, and also there is an advantage that sterilization, etc. can be facilitated.

The discharge pressure of the pump 20 is set to about 3 atm (0.3 MPa) or less. When the infusion solution bag is used, the height difference between the pulsation generator 100 and the liquid level of the infusion solution bag corresponds to the pressure. When the infusion solution bag is used, it is desired to set the height difference so as to attain the pressure of about 0.1 to 0.15 atm (0.01 to 0.015 MPa).

When a surgical operation is carried out by using this fluid ejection device 1, a site gripped by an operator is the pulsation generator 100. Accordingly, it is preferable that the connection tube 25 extending to the pulsation generator 100 is as flexible as possible. Therefore, it is preferable to reduce the pressure by using a flexible and thin tube to the extent that liquid can be fed to the pulsation generator 100. Furthermore, particularly when there is a risk that a trouble of equipment may induce a serious accident like an operation on the brain, it must be avoided that high-pressure fluid is ejected due to cutting or the like of the connection tube 25, and from this viewpoint, the pressure is required to be reduced.

Next, the structure of the pulsation generator 100 according to this embodiment will be described.

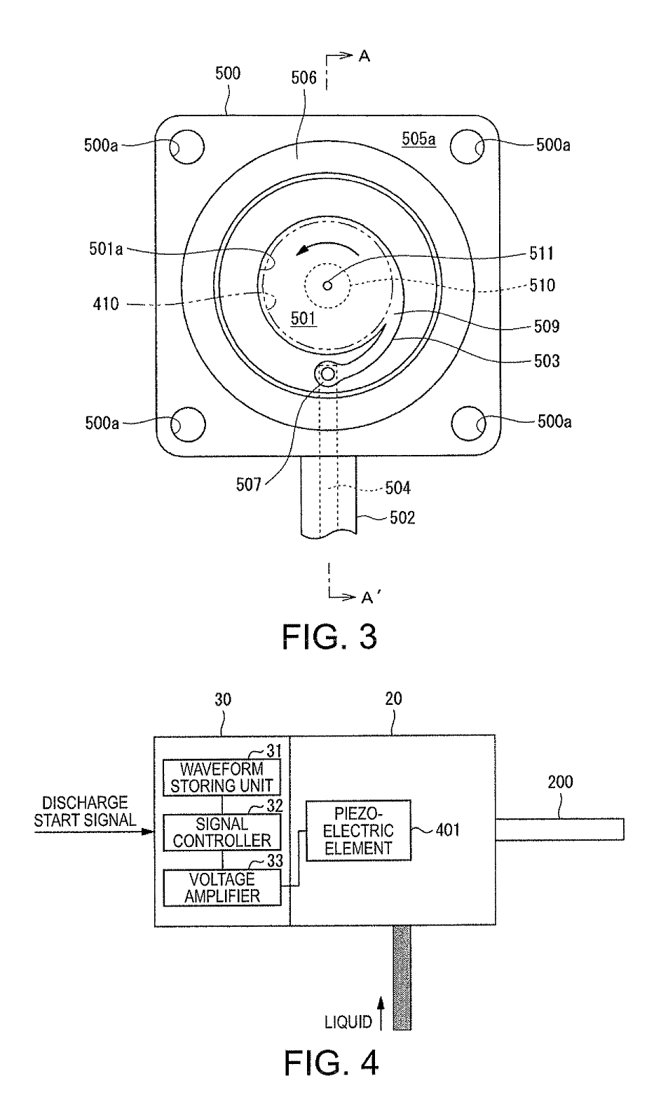

FIGS. 2A and 2B are cross-sectional view and exploded view, respectively, which show the structure of the pulsation generator 100 according to this embodiment. Specifically, FIG. 2A is a cross-sectional view of A-A′ line of FIG. 3 described later.

In FIG. 2A, the pulsation generator 100 contains a pulsation generating unit for generating pulsation of fluid, and the connection fluid channel pipe 200 having a connection fluid channel 201 as a fluid channel for discharging fluid is connected to the pulsation generator 100.

In the pulsation generator 100, an upper case 500 and a lowercase 301 are joined to each other at the confronting faces 302 thereof, and threadably fixed to each other by four fixing screws 600 (partially omitted from the figure). The lower case 301 is designed as a cylindrical member having a flange portion, and one end portion thereof is hermetically sealed by a bottom plate 311. A piezoelectric element 401 is disposed in the internal space of the lower case 301.

The piezoelectric element 401 is a laminate type piezoelectric element, and constitutes an actuator. One end portion of the piezoelectric element 401 is fixed to a diaphragm 400 through an upper plate 411, and the other end portion thereof is fixed to an upper surface 312 of the bottom plate 311.

Furthermore, the diaphragm 400 is formed of a disc-shaped metal thin plate, and fixed in a recess portion 303 of the lower case 301 so that the peripheral edge portion thereof is in close contact with the bottom surface of the recess portion 303. By inputting a driving signal to the piezoelectric element 401, the volume of the fluid chamber 501 is varied through the diaphragm 400 in connection with expansion/contraction of the piezoelectric element 401. A reinforcing plate 410 formed of a disc-shaped metal thin plate having an opening portion at the center portion thereof is laminated and disposed on the upper surface of the diaphragm 400.

The upper case 500 is provided with a recess portion at the center portion of the face thereof which confronts the lower case 301, and a rotational-body shape which is defined by the recess portion and the diaphragm 400 and filled with fluid corresponds to the fluid chamber 501. That is, the fluid chamber 501 is a space surrounded by a sealing face 505 of the recess portion and an inner peripheral side wall 501a of the upper case 500 and the diaphragm 400. An outlet fluid channel 511 is formed substantially at the center portion of the fluid chamber 501. The outlet fluid channel 511 penetrates from the fluid chamber 501 to an end portion of an outlet fluid channel pipe 510 projected from one end face of the upper case 500. The connection portion of the outlet fluid channel 511 to the sealing face 505 of the fluid chamber 501 is smoothly rounded to reduce the fluid resistance.

In this embodiment (see FIGS. 2A and 2B), the shape of the fluid chamber 501 described above is set to a substantially cylindrical shape sealed at both the ends thereof. However, it is not limited to a specific shape, and may be a conical shape, a trapezoidal shape, a semi-spherical shape or the like in side view. For example, if the connection portion between the outlet fluid channel 511 and the sealing face 505 is designed like a funnel, bubbles in the fluid chamber 501 described later are easily discharged.

The connection fluid channel pipe 200 is connected to the outlet fluid channel pipe 510. The connection fluid channel 201 is formed in the connection fluid channel pipe 200, and the diameter of the connection fluid channel 201 is set to be larger than the diameter of the outlet fluid channel 511. Furthermore, the thickness of the pipe portion of the connection fluid channel pipe 200 is set so as to have such rigidity that the pressure pulsation of fluid is not absorbed.

The nozzle 211 is inserted in the tip portion of the connection fluid channel pipe 200. A fluid ejection opening portion 212 is formed in the nozzle 211. The diameter of the fluid ejection opening portion 212 is set to be smaller than the diameter of the connection fluid channel 201.

An inlet fluid channel pipe 502 in which the connection tube 25 for supplying fluid from the pump 20 is inserted is formed in the side surface of the upper case 500, and a connection fluid channel 504 at the inlet fluid channel side is formed in the inlet fluid channel pipe 502. The connection fluid channel 504 intercommunicates with an inlet fluid channel 503. The inlet fluid channel 503 is formed like a groove at the peripheral edge portion of the sealing face 505 of the fluid chamber 501, and intercommunicates with the fluid channel 501.

At the joint face between the upper case 500 and the lower case 301, a packing box 304 is formed at the lower case 301 side and a packing box 506 is formed at the upper case 500 side so as to be far away from the diaphragm 400 in the outer peripheral direction. A ring-shaped packing 450 is mounted in a space defined by the packing box 304 and the packing box 506.

Here, when the upper case 500 and the lower case 301 are assembled with each other, the peripheral edge portion of the diaphragm 400 and the peripheral edge portion of the reinforcing plate 410 are brought into close contact with each other by the peripheral edge portion of the sealing surface 505 of the upper case 500 and the bottom surface of the recess portion 303 of the lower case 301. At this time, the packing 450 is pressed under pressure by the upper case 500 and the lowercase 301, thereby preventing fluid leakage from the fluid chamber 501.

When fluid is discharged, the inside of the fluid chamber 501 is set to a high pressure state of 30 atm (3 MPa) or more, and thus it may be considered that fluid slightly leaks at the respective joint portions of the diaphragm 400, the reinforcing plate 410, the upper case 500 and the lower case 301. However, the fluid leakage at these joint portions is prevented by the packing 450.

When the packing 450 is disposed as shown in FIGS. 2A and 2B, the packing 450 is compressed by the pressure of fluid leaking from the fluid chamber 501 under high pressure, and more strongly pressed against the inner walls of the packing boxes 304 and 506, whereby the leakage of the fluid can be further surely prevented. Accordingly, high pressure increase in the fluid chamber 501 can be kept when the pulsation generator 100 is driven.

Next, the inlet fluid channel 503 formed in the upper case 500 will be described in more detail with reference to the drawings.

FIG. 3 is a plan view showing the inlet fluid channel 503, and shows the upper case 500 when viewed from the contact surface side to the lower case 301. In FIG. 3, the inlet fluid channel 503 is formed like a groove at the peripheral edge portion of the sealing face 505 of the upper case 500. The inlet fluid channel 503 intercommunicates with the fluid chamber 501 at one end portion thereof, and intercommunicates with the connection fluid channel 504 at the other end portion thereof. A fluid pool 507 is formed at the connection portion between the inlet fluid channel 503 and the connection fluid channel 504. The connection portion between the fluid pool 507 and the inlet fluid channel 503 are smoothly rounded to reduce the fluid resistance.

Furthermore, the inlet fluid channel 503 intercommunicates with the fluid chamber 501 so as to extend substantially tangentially with respect to the inner peripheral side wall 501a of the fluid chamber 501. Fluid supplied from the pump 20 (see FIG. 1) under fixed pressure flows along the inner peripheral side wall 501a (the direction indicated by an arrow in FIG. 3), and generates a swirling flow in the fluid chamber 501. The swirling flow is pressed against the inner peripheral side wall 501a by centrifugal force based on swirling, and also bubbles contained in the fluid chamber 501 concentrate on the center portion of the swirling flow.

The bubbles collected at the center portion are excluded from the outlet fluid channel 511. Accordingly, it is preferable that the outlet fluid channel 511 is provided in the neighborhood of the center of the swirling flow, that is, at the center portion of the rotational body shape. Accordingly, in this embodiment, the inlet fluid channel 503 is a swirling flow generator. In FIG. 3, the inlet fluid channel 503 is designed to be curved in plan view. The inlet fluid channel 503 may intercommunicate with the fluid chamber 501 linearly. However, in order to obtain a desired inertance in a narrow space, the fluid channel length of the inlet fluid channel 503 is required to be increased, and thus the inlet fluid channel 503 is curved.

As shown in FIGS. 2A and 2B, the reinforcing plate 410 is disposed between the diaphragm 400 and the peripheral edge portion of the sealing surface 505 at which the inlet fluid channel 503 is formed. The reinforcing plate 410 is mainly provided to enhance the durability of the diaphragm 400. A connection opening portion 509 having a cut-out shape is formed at the connection portion of the inlet fluid channel 503 to the fluid chamber 501. Accordingly, when the diaphragm 400 is driven at a high frequency, stress concentrates in the neighborhood of the connection opening portion 509 and thus fatigue destruction may occur. Therefore, the stress concentration on the diaphragm 400 is prevented from occurring by disposing the reinforcing plate 410 having a continuous opening portion having no cut-out portion.

In this embodiment, the reinforcing plate 410 also has a function of determining the volume of the fluid chamber 501.

Screw holes 500a are formed at four places of the outer peripheral corner portions of the upper case 500, and the upper case 500 and the lower case 301 are threadably joined to each other at these screw hole positions.

As not shown, the reinforcing plate 410 and the diaphragm 400 may be joined to each other and integrally fixed as a laminate body. Adhesive fixing using adhesive agent, solid-layer diffusion joint, welding, etc. may be adopted as a fixing method. It is more preferable that the reinforcing plate 410 and the diaphragm 400 are brought into close contact with each other at the joint surface therebetween.

### Functional Construction of Control System

Next, the functional construction of the control system of the fluid ejection device 1 will be described.

In this embodiment, a driving signal which is input to the piezoelectric element 401 to discharge fluid from the pulsation generator 100 is set to a voltage pulse waveform with which the maximum ejection velocity when fluid is discharged under the state that the nozzle 211 is in the air (hereinafter referred to as “in-air maximum ejection velocity”) is smaller than the maximum ejection velocity when fluid is discharged under the state that the nozzle 211 is in liquid (hereinafter referred to as “in-liquid maximum ejection velocity”). The in-liquid maximum ejection velocity corresponds to an ejection velocity having a capability of incising or cutting out a biomedical tissue (about 20 meters or more per second), and the in-air maximum ejection velocity corresponds to an ejection velocity having no capability of incising or cutting out a biomedical tissue (less than about 20 meters per second).

FIG. 4 is a diagram showing the construction of the control system for the fluid ejection device 1.

In FIG. 4, the control system for the fluid ejection device 1 is mainly constructed by the controller 30, and the controller 30 has a waveform storing unit 31, a signal controller 32 and a voltage amplifier 33. The waveform storing unit 31 stores voltage pulse waveforms of one period representing the driving signal of the piezoelectric element (an original signal before amplified).

In this embodiment, a waveform obtained by cutting out the amplitude at the positive side of a sine wave (hereinafter referred to as “basic pulse waveform”) may be used as the voltage pulse waveform, or a waveform obtained by gradually diminishing the amplitude from the apex of the basic pulse waveform till the rise-up point of the next basic pulse waveform (hereinafter referred to as “composite wave”) may be used as the voltage pulse waveform. In this case, the description will be made in the case of use of the composite wave.

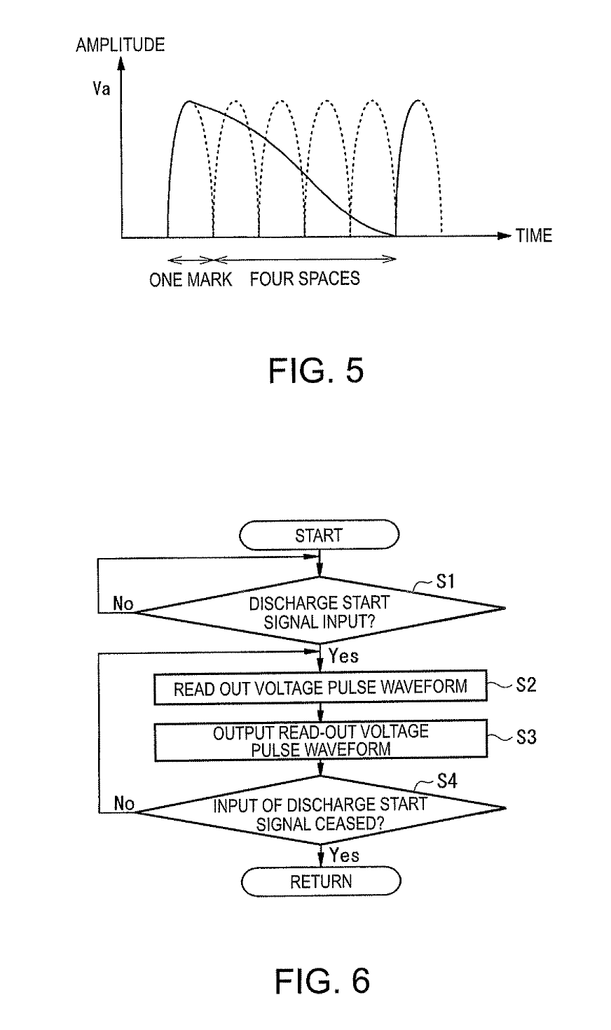

FIG. 5 is a schematic diagram showing the voltage pulse waveform stored in the waveform storing unit 31. The waveform storing unit 31 stores the voltage pulse waveform as a digital value, however, the voltage pulse waveform is illustrated in the analog style for convenience's sake of description in FIG. 5.

As shown in FIG. 5, the waveform storing unit 31 stores a voltage pulse waveform of a composite wave whose amplitude rises up to the apex (amplitude Va) of the basic pulse waveform at the start portion of the period and then gradually diminishes from that apex till the end time point of one period. The voltage pulse waveform of the composite wave shown in FIG. 5 is constructed on the basis of a waveform in which one basic pulse waveform is disposed at the head and spaces (amplitude-zero state) corresponding to the periods of four basic pulse waveforms indicated by undulating lines are set.

That is, the voltage pulse waveform of the composite wave shown in FIG. 5 is the waveform in which spaces (four spaces) corresponding to four basic pulse waveforms are inserted with respect to one substantive basic pulse waveform (one mark). Therefore, the voltage pulse waveform having such a period is referred to as 1m4s (1 mark 4 spaces).

In this embodiment, in the case of the voltage pulse waveform of 1m4s, the maximum ejection velocity when fluid is discharged under the state that the nozzle 211 is in the air (in-air maximum ejection velocity) is smaller than the maximum ejection velocity when fluid is discharged under the state that the nozzle 211 is in liquid (in-liquid maximum ejection velocity). When the period is lengthened to the same level as the period of 1m13s, the respective maximum ejection velocities in the case where the nozzle 211 is in the air and the case where the nozzle 211 is in liquid are equal to the same level.

The period of the voltage pulse waveform with which the in-air maximum ejection velocity and the in-liquid maximum ejection velocity are different from each other is varied in accordance with the characteristic of the fluid channel comprising the connection fluid channel pipe 200 and the nozzle 211. However, in the case of voltage pulse waveforms having the same amplitude, the boundary between the voltage pulse waveforms can be found by gradually setting a long period (low frequency) signal to a short period (high frequency) signal.

Accordingly, the boundary value of the signal period with which the in-air maximum ejection velocity and the in-liquid maximum ejection velocity are significantly different from each other can be obtained as an experiment value which is obtained by using the fluid ejection device 1. In the fluid ejection device 1 according to this embodiment, the boundary period of the voltage pulse waveform with which the in-air maximum ejection velocity and the in-liquid maximum ejection velocity are different from each other is equal to 1m5s, and the voltage pulse waveform of 1m4s which is shorter than the period of 1m5s is used as the driving signal.

The signal controller 32 has a function of controlling the whole fluid ejection device 1. Specifically, the signal controller 32 executes discharge control processing described later, and when a discharge start signal for instructing to discharge fluid is input from a foot switch (not shown) or the like, the signal controller 32 reads out a voltage pulse waveform stored in the waveform storing unit 31 and outputs a digital value representing the read-out voltage pulse waveform (the original signal before amplification) to the voltage amplifier 33.

The discharge start signal is a signal which is input by operator's operation of a switch to start ejection of fluid from the fluid ejection device 1 when a target site is incised or the like. The voltage amplifier 33 is constructed by an amplifier having a push-pull circuit, for example, and it converts a digital signal of the voltage pulse waveform input from the signal controller 32 to an analog signal, amplifies the analog signal concerned and then applies the amplified analog signal to the piezoelectric element 401 of the pulsation generator 100.

### Operation

Next, the operation of this embodiment will be described.

### Control Operation of Fluid Ejection Device 1

FIG. 6 is a flowchart showing the discharge control processing executed by the signal controller 32. The discharge control processing is started to be executed together with power-on of the fluid ejection device 1, and it is repetitively executed during the power-on state of the fluid ejection device 1.

In FIG. 6, when the discharge control processing is started, the signal controller 32 determines whether the discharge start signal is input or not (step S1), and when it is determined that no discharge start signal is input, the signal controller 32 repeats the processing of step S1.

On the other hand, it is determined in step S1 that the discharge start signal is input, the voltage pulse waveform stored in the waveform storing unit 31 is read out (step S2). Then, the signal controller 32 outputs the digital signal representing the read-out voltage pulse waveform to the voltage amplifier 33 (step S3).

Subsequently, the signal controller 32 determines whether the input of the discharge start signal is ceased or not (step S4). When it is determined that the input of the discharge start signal is not ceased, the signal controller 32 shifts to the processing of the step S2. When it is determined that the input of the discharge start signal is ceased, the signal controller 32 repeats the discharge control processing.

### Operation of Whole Fluid Ejection Device 1

Subsequently, the operation of the whole fluid ejection device 1 when the above control operation is executed will be described. The fluid discharge of the pulsation generator 100 according to this embodiment is executed on the basis of the difference between the inertance L1 (also called as composite inertance L1) at the inlet fluid channel side and the inertance L2 (also called as composite inertance L2) at the outlet fluid channel side.

First, the inertance will be described.

The inertance L is represented by L=ρ×h/S, wherein ρ represents the density of fluid, S represents the cross-sectional area of the fluid channel and h represents the length of the fluid channel. When the pressure difference of the fluid channel is represented by ΔP and the flow amount of the fluid flowing in the fluid channel is represented by Q, the relational expression: ΔP=L×dQ/dt is derived by transforming the dynamic equation in the fluid channel by using the inertance L.

That is, the inertance L represents the degree of an effect applied to the time-variation of the flow amount. The time-variation of the flow amount is smaller as the inertance L is larger, and the time-variation of the flow amount is larger as the inertance L is smaller.

Furthermore, the composite inertance concerning the parallel connection of plural fluid channels and the serial connection of plural fluid channels different in shape can be calculated by combining the inertances of individual fluid channels as in the case of the parallel connection or serial connection of inductances in an electrical circuit.

The connection fluid channel 504 is designed to be sufficiently larger in diameter than the inlet fluid channel 503, and thus the inertance L1 at the inlet fluid channel side is calculated in the range of the inlet fluid channel 503. At this time, the connection tube for connecting the pump 20 and the inlet fluid channel has flexibility, and thus it may be omitted from the calculation of the inertance L1.

The diameter of the connection fluid channel 201 is remarkably larger than that of the outlet fluid channel, and the thickness of the pipe portion (pipe wall) of the connection fluid channel pipe 200 is small, so that the effect on the inertance L2 at the outlet fluid channel side is minor. Accordingly, the inertance L2 at the outlet fluid channel side may be replaced with the inertance of the outlet fluid channel 511. The thickness of the pipe wall of the connection fluid channel pipe 200 is set to provide sufficient rigidity to the pressure propagation of fluid.

In this embodiment, the fluid channel length and cross-sectional area of the inlet fluid channel 503 and the fluid channel length and cross-sectional area of the outlet fluid channel 511 are set so that the inertance L1 at the inlet fluid channel side is larger than the inertance L2 at the outlet fluid channel side.

The operation of the pulsation generator 100 will be described.

The fluid is supplied to the inlet fluid channel 503 under fixed liquid pressure at all times by the pump 20. As a result, when the piezoelectric element 401 does not operate, the fluid flows in the fluid chamber 501 due to the difference between the discharge force of the pump 20 and the overall fluid resistance value of the inlet fluid channel side. Here, it is assumed that the discharge start signal is input to the controller 30 by operating the foot switch or the like.

At this time, the voltage pulse waveform is read out from the waveform storing unit 31 and then applied through the voltage amplifier 33 to the piezoelectric element 401 by the signal controller 32 executing the discharge control processing. When the voltage pulse waveform is input to the piezoelectric element 401 and the piezoelectric element 401 expands drastically, the pressure in the fluid chamber 501 would quickly increase and reach several tens atm if the inertances L1 and L2 at the inlet fluid channel side and the outlet fluid channel side are sufficiently large.

This pressure is remarkably larger than the pressure applied to the inlet fluid channel 503 by the pump 20. Therefore, the flow-in of the fluid from the inlet fluid channel side into the fluid chamber 501 is reduced by the pressure concerned, and the flow-out of the fluid from the outlet fluid channel 511 is increased. Accordingly, the check valve provided at the inlet fluid channel side is not required.

However, the inertance L1 of the inlet fluid channel 503 is larger than the inertance L2 of the outlet fluid channel 511, and thus the increase amount of the fluid discharged from the outlet fluid channel is larger than the decrease amount of the flow amount of the fluid from the inlet fluid channel 503 into the fluid chamber 501, so that pulsed fluid discharge, that is, pulsation flow occurs in the connection fluid channel 201. The pressure variation at this discharge time propagates in the connection fluid channel pipe 200, and fluid is ejected from the fluid ejection opening portion 212 of the nozzle 211 at the tip.

Here, the diameter of the fluid ejection opening portion 212 of the nozzle 211 is smaller than the diameter of the outlet fluid channel 511, and thus the fluid is ejected as high-pressure and high-velocity pulsed liquid droplets.

Furthermore, the decrease of the flow-in amount of the fluid from the inlet fluid channel 503 and the increase of the flow-out amount of the fluid from the outlet fluid channel 511 mutually interacts with each other, so that the inside of the fluid chamber 501 is set to a vacuum state immediately after the pressure is increased. As a result, the flow of the fluid in the inlet fluid channel 503 which directs into the fluid chamber 501 at a velocity equal to that before the piezoelectric element 401 is operated is restored after lapse of a fixed time by both the pressure of the pump 20 and the vacuum state in the fluid chamber 501.

After the fluid flow of the fluid in the inlet fluid channel 503 is restored, the pulsation flow from the nozzle 211 can be continually ejected when the piezoelectric element 401 expands.

In the discharge operation of the voltage pulse waveform described above, immediately after the fluid is ejected from the fluid ejection opening portion 212 of the nozzle 211, the fluid at the tip portion of the connection fluid channel pipe 200 is discharged with following the just-before ejected fluid due to the viscosity of the fluid and the inertance of the connection fluid channel 201. Therefore, when fluid is ejected in the air, a portion at which air is temporarily contaminated in fluid (hereinafter referred to as “gas-liquid mixture portion”) occurs at the backside position of the nozzle 211 in the connection fluid channel pipe 200 after the fluid is ejected.

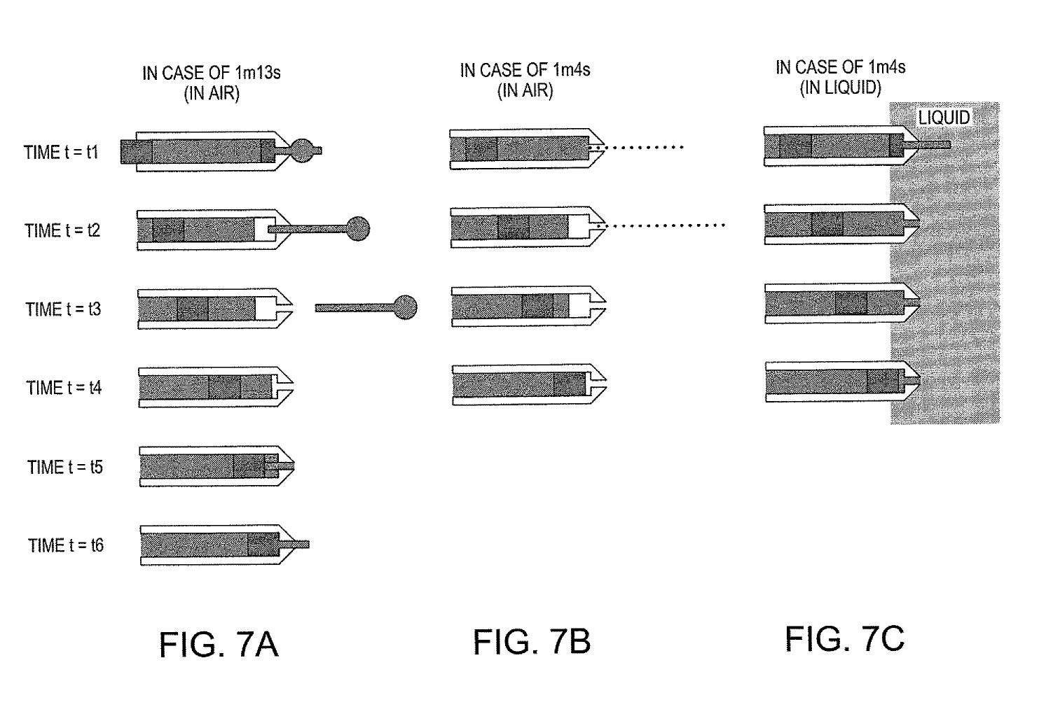

FIGS. 7A to 7C are diagrams showing the state of the connection fluid pipe 200 when fluid is ejected. FIG. 7A shows a case where the piezoelectric element 401 is driven with a voltage pulse waveform of 1m13s in the air, FIG. 7B shows a case where the piezoelectric element 401 is driven with a voltage pulse waveform of 1m4s in the air, and FIG. 7C shows a case where the piezoelectric element 401 is driven with a voltage pulse waveform of 1m4s in liquid. In FIGS. 7A to 7C, the state of the connection fluid channel pipe 200 is successively shifted in time order of t=t1 to t6.

As shown in FIG. 7A, when the piezoelectric element 401 is driven with the voltage pulse waveform (1m13s) of a long period in the air, the gas-liquid mixture portion occurring at the backside of the nozzle 211 is gradually pushed out from the nozzle 211 by the pressure of the fluid by the pump 20 or the like within the timing of the space (the zero-amplitude period). The next ejection is carried out after the gas-liquid mixture portion vanishes and the subsequent pulse is enabled to push the fluid to the fluid ejection opening portion 212 under high pressure.

On the other hand, as shown in FIG. 7B, when the piezoelectric element 401 is driven with the voltage pulse waveform (1m4s) of a short period in the air, the fluid resistance value when the fluid is ejected to the outside of the nozzle 211 is smaller than that in the case where the fluid is ejected in liquid, and thus the discharge is continued for a long time. Therefore, although the gas-liquid mixture portion occurring at the backside of the nozzle 211 does not perfectly vanish within the timing of the space, the subsequent pulse reaches the gas-liquid mixture portion. Therefore, the next ejection is executed under the state that the fluid cannot be pressed out under high pressure.

Accordingly, when the piezoelectric element 401 is driven with the voltage pulse waveform of the long period, the fluid is ejected at a high velocity at which a biomedical tissue can be incised or the like in the air.

On the other hand, when the piezoelectric element 401 is driven with the voltage pulse waveform of the short period, the fluid is ejected at the velocity at which the biomedical tissue cannot be incised or the like in the air.

Furthermore, as shown in FIG. 7C, no gas-liquid mixture portion occurs in liquid. Therefore, even when the piezoelectric element 401 is driven with the voltage pulse waveform of the short period, the fluid can be ejected at a high velocity at which a biomedical tissue can be incised or the like. The voltage pulse waveform of the short period in this embodiment is set within a period range in which the fluid ejection velocity in liquid (the in-liquid maximum ejection velocity) is the same level as the fluid ejection velocity when the voltage pulse waveform of the long period is used in the air (the in-air maximum ejection velocity).

That is, the voltage pulse waveform of the driving signal in this embodiment is set to the period at which the fluid ejection velocity in the air is in the maximum ejection velocity in the range in which the biomedical tissue cannot be incised or the like due to the effect of the gas-liquid mixture portion, however, the fluid ejection velocity in liquid is in the maximum ejection velocity in the range in which the biomedical tissue can be incised or the like.

As described above, in the fluid ejection device 1 according to this embodiment, the voltage pulse waveform with which the in-air maximum ejection velocity is smaller than the in-liquid maximum ejection velocity is applied as the driving signal of the piezoelectric element 401. Therefore, when the nozzle 211 is in liquid, that is, when a biomedical tissue is incised or cut out, fluid is ejected at an ejection velocity having a capability of incision, etc. However, when the nozzle 211 is in the air, that is, when a biomedical tissue is not incised or the like, fluid is ejected at an ejection velocity having no capability of incision, etc.

Accordingly, when an operator receives/delivers the handpiece from/to another operator or the like, fluid would be merely ejected at the ejection velocity having no capability of incision, etc. even if the fluid is erroneously ejected under the state that the nozzle is not in close contact with a target site. Therefore, the effect when the fluid spatters to a surrounding area or the fluid is ejected to an unexpected site can be suppressed. That is, in the fluid ejection device 1 according to this embodiment, the discharge control of fluid can be more properly performed.

In FIGS. 7A to 7C, the description is made in the case where the gas-liquid mixture portion occurs at the backside of the nozzle 211 at the fluid ejection time. However, it may be considered that the gas-liquid mixture portion may occur not only at the backside of the nozzle 211, but also at the center portion or the like of the connection fluid channel pipe 200, or at any place from the exit of the fluid chamber 501 till the nozzle 211 in accordance with the characteristic of the fluid channel.

Even in such a case, by applying to the piezoelectric element 401 the voltage pulse waveform with which the in-air maximum ejection velocity is smaller than the in-liquid maximum ejection velocity as in the embodiment, the effect when fluid is ejected in the air due to erroneous operation can be suppressed, and the fluid discharge control can be more properly performed.

What is claimed is:

1. A fluid ejection device comprising:
a fluid chamber in which fluid flows;
a volume varying unit having a pressure generating element that varies the volume of the fluid chamber on the basis of a driving signal;
an inlet fluid channel and an outlet fluid channel intercommunicating with the fluid chamber;
a fluid channel pipe having a connection fluid channel that intercommunicates with the outlet fluid channel at a first end thereof and intercommunicates with a fluid ejection port at a second end thereof;
a fluid supply unit that supplies fluid to the inlet fluid channel; and
a controller that applies a driving signal to the pressure generating element and controls variation of the volume of the fluid chamber by the volume varying unit, wherein the controller applies a driving signal to the pressure generating element so that a maximum ejection velocity of fluid in liquid is larger than a maximum ejection velocity of fluid in the air.

2. The fluid ejection device according to claim 1, wherein the controller applies as the driving signal voltage pulse waveforms whose period is set so that the maximum ejection velocity of fluid in liquid is larger than the maximum ejection velocity of fluid in the air.

3. The fluid ejection device according to claim 1, wherein the controller applies to the pressure generating element the driving signal with which the maximum ejection velocity of the fluid in the air is less than 20 meters per second.

4. A fluid ejection device comprising:
a fluid chamber in which fluid flows;
a volume varying unit having a pressure generating element that varies the volume of the fluid chamber on the basis of a driving signal;
an inlet fluid channel and an outlet fluid channel intercommunicating with the fluid chamber;
a fluid channel pipe having a connection fluid channel that intercommunicates with the outlet fluid channel at a first end thereof and intercommunicates with a fluid ejection port at a second end thereof;
a fluid supply unit that supplies fluid to the inlet fluid channel; and
a controller that applies a driving signal to the pressure generating element and controls variation of the volume of the fluid chamber by the volume varying unit, wherein the controller applies a driving signal to the pulsation generator so that fluid and gas are mixed with each other to form a gas-liquid mixture portion in the connection fluid channel pipe due to a fluid discharge executed immediately before the fluid is discharged in the air, and the fluid discharge is subsequently executed under a state that the gas-liquid mixture portion remains.

5. The fluid ejection device according to claim 4, wherein the controller applies the driving signal so that the gas-liquid mixture portion is formed at the back side of the fluid ejection port of the connection fluid channel pipe when the fluid is discharged in the air.

6. A fluid ejection method comprising:
ejecting fluid to which pressure is applied by a pressure generating element so that a maximum ejection velocity of the fluid in liquid is larger than a maximum ejection velocity of the fluid in the air when the fluid is ejected through a fluid channel from a fluid ejection port.
